(19) United States
(12) Patent Application Publication
(10) Pub. No.: US 2022/0257653 A1
(43) Pub. Date: Aug. 18, 2022

(54) CHIMERIC ANTIGEN RECEPTORS CONTAINING GLYPICAN 2 BINDING DOMAINS

(71) Applicants: The Children's Hospital of Philadelphia, Philadelphia, PA (US); THE UNITED STATES OF AMERICA, AS REPRESENTED BY THE SECRETARY, DEPARTMENT OF HEALTH AND HUMAN SERVIC, Bethesda, CA (US); THE BOARD OF TRUSTEES OF THE LELAND STANFORD JUNIOR UNIVERSITY, Standford, CA (US)

(72) Inventors: Kristopher BOSSE, Philadelphia, PA (US); John MARIS, Philadelphia, PA (US); David BARRETT, Philadelphia, PA (US); Jessica FOSTER, Wynnewood, PA (US); Dimiter DIMITROV, Frederick (UD); Crystal MACKALL, Redwood City, CA (US); Robbie MAJZNER, Palo Alto, CA (US); ZHONGYU ZHU, Frederick, MD (US); Sabine HEITZENEDER, Redwood City, CA (US); Dontcho V. JELEV, ROCKVILLE, MD (US)

(73) Assignees: The Children's Hospital of Philadelphia, Philadelphia, PA (US); THE UNITED STATES OF AMERICA, AS REPRESENTED BY THE SECRETARY, DEPARTMENT OF HEALTH AND HUMAN SERVIC, Bethesda, MD (US); THE BOARD OF TRUSTEES OF THE LELAND STANFORD JUNIOR UNIVERSITY, Standford, CA (US)

(21) Appl. No.: 17/597,580

(22) PCT Filed: Jul. 17, 2020
(86) PCT No.: PCT/US2020/042469

§ 371 Date: Jan. 12, 2022

Related U.S. Application Data
(60) Provisional application No. 62/876,483, filed on Jul. 19, 2019.

(51) Int. Cl. A61K 35/17; C07K 14/725; C07K 16/30; C07K 14/705; A61P 35/00

(52) U.S. Cl. CPC A61K 35/17; C07K 14/7051; C07K 16/3053; C07K 14/70517; A61K 38/00; C07K 14/70578; A61P 35/00; C07K 2317/24; C07K 14/70521

(57) ABSTRACT

The present disclosure is directed to chimeric antigen receptors binding to Glypican 2, nucleic acids encoding the same, and cells expressing the same, and methods of using such cells to treat cancers that express or overexpress the Glypican 2 antigen.

### PRIORITY CLAIM

This application claims benefit of priority to U.S. Provisional Application Ser. No. 62/876,483, filed Jul. 19, 2019, the entire contents of which are hereby incorporated by reference.

### STATEMENT REGARDING FEDERAL FUNDING

This invention was made with government support under Grant Number NCI U54 CA232568-01 awarded by the National Institues of Health. The government has certain rights in the invention.

Pursuant to 37 C.F.R. § 1.821(c), a sequence listing is submitted herewith as an ASCII compliant text file named “CHOPP0034WO.txt”, created on Jul. 17, 2020 and having a size of ˜27 kilobytes. The content of the aforementioned file is hereby incorporated by reference in its entirety.

### BACKGROUND

### 1. Field

The present disclosure relates generally to the fields of medicine, oncology and immunotherapeutics. More particularly, it concerns the development of chimeric antigen receptors immunoreagents with binding specificity for glypican 2 (GPC2) and their use in treating GPC2-positive cancers.

### 2. Related Art

Children with high-risk neuroblastoma have a poor prognosis despite intensive multimodal chemoradiotherapy. While monoclonal antibodies targeting the disialoganglioside GD2 improve outcomes in neuroblastoma, this therapy is associated with significant “on target-off tumor” toxicities. Thus, a major challenge remains in identifying novel cell surface molecules that meet the stringent criteria for modern immunotherapeutics, including unique tumor expression compared to normal childhood tissues, and preferably that these cell surface molecules be required for tumor sustenance.

A number of biopharmaceuticals for the treatment of diseases or health disorders are under current development by pharmaceutical and biotechnology companies. For example, in cancer immunotherapy, the development of agents that activate T cells of the host's immune system to prevent the proliferation of or kill cancer cells, has emerged as a promising therapeutic approach to complement existing standards of care. Adoptive transfer of T cells, especially chimeric antigen receptor (CAR)-engineered T cells, has emerged as another promising approach in cancer immunotherapy. Unlike naturally occurring T cell receptors, CARs can directly recognize their target antigens without restrictions imposed by major histocompatibility complex (MHC) molecules and can potentially mediate high levels of cell-killing activity. One common method is to genetically engineer T cells ex vivo to express CARs which can recognize target antigens without the need for MHC presentation. These CAR-T cells have the potential to generate very high levels of anti-tumor activity, but they may also display increased off-target cell killing of CAR-T cells. Accordingly, there remains an urgent need for alternative approaches to minimize such side-effect and to complement existing approaches for immunotherapy.

### SUMMARY

Thus, in accordance with the present disclosure, there is provided a chimeric antigen receptor comprising (i) an ectodomain comprising single-chain antibody variable region fragment (scFv) region comprising a variable heavy chain (VH) and a variable light chain (VL) that binds selectively to Glypican 2, (ii) a transmembrane domain; and (iii) an endodomain, wherein said endodomain comprises a signal transduction function when said scFv is engaged with Glypican 2.

The receptor may be characterized by VH and VL sequences of SEQ ID NOS: 5 and 6, respectively; by VH and VL sequences of SEQ ID NOS: 7 and 8, respectively, or by VH and VL sequences of SEQ ID NOS: 9 and 10, respectively.

The scFv may be characterized by VH and VL sequences having 80% homology to SEQ ID NOS: 5 and 6, respectively, and having VH CDRs of SEQ ID NOS: 11-13 and VL CDRs of SEQ ID NOS: 14-16; or by VH and VL sequences having 80% homology to SEQ ID NOS: 7 and 8, respectively, and having VH CDRs of SEQ ID NOS: 17-19 and VL CDRs of SEQ ID NOS: 20-22; or by VH and VL sequences having 80% homology to SEQ ID NOS: 9 and 10, respectively, and having VH CDRs of SEQ ID NOS: 23-25 and VL CDRs of SEQ ID NOS: 26-28.

The receptor may be characterized by VH and VL sequences having 90% homology to SEQ ID NOS: 5 and 6, respectively, and having VH CDRs of SEQ ID NOS: 11-13 and VL CDRs of SEQ ID NOS: 14-16; or by VH and VL sequences having 90% homology to SEQ ID NOS: 7 and 8, respectively, and having VH CDRs of SEQ ID NOS: 17-19 and VL CDRs of SEQ ID NOS: 20-22; or by VH and VL sequences having 90% homology to SEQ ID NOS: 9 and 10, respectively, and having VH CDRs of SEQ ID NOS: 23-25 and VL CDRs of SEQ ID NOS: 26-28.

The receptor may comprises a sequence selected from SEQ ID NOS: 1, 2 and 3; or may comprise a sequence that is 80% homologous to SEQ ID NOS: 1, 2 or 3, and having VH CDRs of SEQ ID NOS: 11-13, 17-19 and 23-25, respectively, and VL CDRs of SEQ ID NOS: 14-16, 20-22 and 26-28, respectively, or may comprise a sequence that is 90% homologous to SEQ ID NOS: 1, 2 or 3, and having VH CDRs of SEQ ID NOS: 11-13, 17-19 and 23-25, respectively, and VL CDRs of SEQ ID NOS: 14-16, 20-22 and 26-28, respectively.

The transmembrane and endodomains may be derived from the same molecule. The endodomain may be comprise a CD3-zeta domain or a high affinity FcεRI. The scFv may comprise a flexible linker disposed between said VH and VL, such as wherein the flexible linker is from CD8α, Ig or SEQ ID NO: 4. The scFv may be arranged VH-linker-VL or VL-linker-VH.

Also provided is a nucleic acid encoding the chimeric antigen receptor as defined above, such as an mRNA or a DNA, or a cell expressing the chimeric antigen receptor as defined above, such as a prokaryotic cell or a eukaryotic cell, and in particular an engineered T cell.

In another embodiment, there is provided A method of treating a subject having cancer that expresses or overexpress Glypican 2 comprising administering to said subject a chimeric antigen receptor as defined above, the nucleic acid as defined above, or the cell as defined above, such as a T cell, such as a T cell that is autologous to said subject.

The method may further comprise administering to said subject a second anti-cancer therapy. The second cancer therapy may be radiation, chemotherapy, radiotherapy, hormonal therapy, immunotherapy, toxin therapy or surgery. The immunotherapy may be a checkpoint inhibitor therapy. The second cancer therapy may be administered at the same time as said receptor, nucleic acid or cell, or may be administered before or after said receptor, nucleic acid or cell. The second cancer therapy may be administered more than once. The receptor, nucleic acid or cell may be administered more than once.

The cancer may be drug-resistant, metastatic or recurrent. The subject may be a human or non-human mammal. The cancer may be a pediatric cancer or an adult cancer. The cancer may be a leukemia, such as a leukemia selected from the group consisting of acute lymphoblastic leukemia (ALL), acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), and hairy cell leukemia.

The cancer may be a solid tumor cancer, such as a lung cancer, liver cancer, pancreatic cancer, stomach cancer, colon cancer, kidney cancer, brain cancer, head and neck cancer, breast cancer, skin cancer, rectal cancer, uterine cancer, cervical cancer, ovarian cancer, testicular cancer, skin cancer, or esophageal cancer. The cancer may also comprise a sarcoma cell, a rhabdoid cancer cell, a neuroblastoma cell, retinoblastoma cell, or a medulloblastoma cell. The cancer may be uterine carcinosarcoma (UCS), brain lower grade glioma (LGG), thymoma (THYM), testicular germ cell tumors (TGCT), glioblastoma multiforme (GBM) and skin cutaneous melanoma (SKCM), liver hepatocellular carcinoma (LIHC), uveal melanoma (UVM), kidney chromophobe (KICH), thyroid cancer (THCA), kidney renal clear cell carcinoma (KIRC), kidney renal papillary cell carcinoma (KIRP), stomach adenocarcinoma (STAD), cholangiocarcinoma (CHOL), adenoid cystic carcinoma (ACC), prostate adenocarcinoma (PRAD), pheochromocytoma and paraganglioma (PCPG), DLBC, lung adenocarcinoma (LUAD), head-neck squamous cell carcinoma (HNSC), pancreatic adenocarcinoma (PAAD), breast cancer (BRCA), mesothelioma (MESO), colon and rectal adenocarcinoma (COAD), rectum adenocarcinoma (READ), esophageal carcinoma (ESCA), ovarian cancer (OV), lung squamous cell carcinoma (LUSC), bladder urothelial carcinoma (BLCA), sarcoma (SARC), or uterine corpus endometrial carcinoma (UCEC).

Also provided is an isolated nucleic acid molecule encoding a chimeric antigen receptor (CAR), wherein the CAR comprises an antigen binding domain, a flexible hinge domain, a transmembrane domain, a costimulatory signaling region, and an intracellular signaling domain, and wherein the antigen binding domain binds selectively to a cancer cell-associated Glypican 2 (GPC2). The antigen binding domain may comprise an antibody or an antigen-binding fragment thereof. The antigen-binding fragment may be a Fab, a single-chain variable fragment (scFv), or a single-domain antibody. The encoded antigen binding domain may comprises (a) a heavy chain variable domain comprising the amino acid sequence of SEQ ID NO: 30 and a light chain variable domain comprising the amino acid sequence of SEQ ID NO: 32; or (b) a heavy chain variable domain comprising the amino acid sequence of SEQ ID NO: 34, and a light chain variable domain comprising the amino acid sequence of SEQ ID NO: 36, or (c) a heavy chain variable domain comprising the amino acid sequence of SEQ ID NO: 38 and a light chain variable domain comprising the amino acid sequence of SEQ ID NO: 40.

The encoded antigen binding domain may comprise (a) a heavy chain variable domain including a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, and a light chain variable domain including a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; or (b) a heavy chain variable domain including a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, and a light chain variable domain including a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; or (c) a heavy chain variable domain including a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, and a light chain variable domain including a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

The encoded antigen binding domain may comprise a heavy chain variable domain comprising the amino acid sequence of SEQ ID NO: 30 and a light chain variable domain comprising the amino acid sequence of SEQ ID NO: 32; and the C-terminus of the light chain variable domain may be fused to the N-terminus of a heavy chain variable domain by a flexible linker. The linker may be a peptide linker, such as at least 15 amino acids in length, and/or the peptide linker may be a glycine-serine linker. The isolated nucleic acid molecule may have (a) the flexible hinge domain from CD8α, CD28, or an immunoglobulin (Ig), (b) the transmembrane domain comprising CD28 transmembrane domain, (c) the costimulatory signaling region comprising a domain from CD28, 41BB (CD137), OX40, or ICOS, and (d) the intracellular signaling domain comprising a CD3-zeta domain or a high affinity FcεRI.

In another embodiment, there is provided a chimeric antigen receptor (CAR) polypeptide, wherein (a) the CAR comprises an antigen binding domain, a flexible hinge domain, a transmembrane domain, a costimulatory signaling region, and an intracellular signaling domain; and (b) the antigen binding domain binds selectively to cancer cell-associated Glypican 2 (GPC2). The antigen-binding fragment may be a Fab, a single-chain variable fragment (scFv), or a single-domain antibody.

The encoded antigen binding domain may comprise (a) a heavy chain variable domain comprising the amino acid sequence of SEQ ID NO: 30 and a light chain variable domain comprising the amino acid sequence of SEQ ID NO: 32; or (b) a heavy chain variable domain comprising the amino acid sequence of SEQ ID NO: 34, and a light chain variable domain comprising the amino acid sequence of SEQ ID NO: 36, or (c) a heavy chain variable domain comprising the amino acid sequence of SEQ ID NO: 38 and a light chain variable domain comprising the amino acid sequence of SEQ ID NO: 40.

The encoded antigen binding domain may comprise (a) a heavy chain variable domain including a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, and a light chain variable domain including a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; or (b) a heavy chain variable domain including a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, and a light chain variable domain including a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; or (c) a heavy chain variable domain including a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, and a light chain variable domain including a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

The chimeric antigen receptor polypeptide may comprise (a) an encoded antigen binding domain comprising a heavy chain variable domain comprising the amino acid sequence of SEQ ID NO: 30 and a light chain variable domain comprising the amino acid sequence of SEQ ID NO: 32; and (b) a C-terminus of the light chain variable domain fused to the N-terminus of a heavy chain variable domain by a flexible linker.

Also provided is a genetically modified T cell comprising a nucleic acid sequence encoding a chimeric antigen receptor (CAR), or a genetically modified T cell comprising the isolated nucleic acid molecule as defined herein, or comprising the chimeric antigen receptor as defined herein. The genetically modified T cell may be characterized as:
(a) a CAR that induces interferon γ and Interleukin-2 secretion, and
(b) exhibiting cytotoxicity toward a GPC2 expressing cancer when the genetically modified T cell is exposed to the cancer cell-associated GPC2.
   The GPC2 expressing cancer may be selected from the group consisting of sarcoma cell, a rhabdoid cancer cell, a neuroblastoma cell, retinoblastoma cell, or a medulloblastoma cell, uterine carcinosarcoma (UCS), brain lower grade glioma (LGG), thymoma (THYM), testicular germ cell tumors (TGCT), glioblastoma multiforme (GBM) and skin cutaneous melanoma (SKCM), liver hepatocellular carcinoma (LIHC), uveal melanoma (UVM), kidney chromophobe (KICH), thyroid cancer (THCA), kidney renal clear cell carcinoma (KIRC), kidney renal papillary cell carcinoma (KIRP), stomach adenocarcinoma (STAD), cholangiocarcinoma (CHOL), adenoid cystic carcinoma (ACC), prostate adenocarcinoma (PRAD), pheochromocytoma and paraganglioma (PCPG), DLBC, lung adenocarcinoma (LUAD), head-neck squamous cell carcinoma (HNSC), pancreatic adenocarcinoma (PAAD), breast cancer (BRCA), mesothelioma (MESO), colon and rectal adenocarcinoma (COAD). rectum adenocarcinoma (READ), esophageal carcinoma (ESCA), ovarian cancer (OV), lung squamous cell carcinoma (LUSC), bladder urothelial carcinoma (BLCA), sarcoma (SARC), or uterine corpus endometrial carcinoma (UCEC).

Also provided is a method of making a genetically modified T cell comprising transducing the immune effector cell with the chimeric antigen receptor as defined herein. Also provided is a method of providing an anti-tumor immunity in a mammal, comprising administering to the mammal an effective amount of a population of genetically modified T cells as defined herein. Also provided is a method of treating a mammal having a disease associated with overexpression of a GPC2, the method comprising administering to the mammal an effective amount of a population of a genetically modified T cells as defined herein.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Heavy and light chain amino acid and nucleic acid sequences for human antibody m201. CDRs shown in bold italics.

FIG. 2. Heavy and light chain amino acid and nucleic acid nucleic sequences for human antibody m202. CDRs shown in bold italics.

FIG. 3. Heavy and light chain amino acid and nucleic acid sequences for human antibody m203. CDRs shown in bold italics.

FIG. 4. GPC2 is expressed in a subset of pediatric brain tumors. RNA sequencing data from the Childhood Brain Tumor Tissue Consortium (CBTTC) including 1110 samples.

FIGS. 5A-C. In vitro validation of GPC2 RNA CAR T cell binding and persistence. (FIG. 5A) GPC2 RNA CAR specific binding to GPC2 of the four GPC2 RNA CAR T cell constructs, measured by flow cytometry. (FIG. 5B) CAR persistence over time for each construct, measured by flow cytometry. (FIG. 5C) Negative checkpoint regulator expression PD1 and Lag3 for each construct at four days after transfection.

FIGS. 6A-D. D3V3 and D3V4 mRNA GPC2 CAR T cells produce strongest cytotoxicity in vitro. (FIG. 6A) Cytotoxicity of the four GPC2 CAR T cell constructs against SMS-SAN, endogenously high GPC2 expressing neuroblastoma cell line. E:T ratio 10:1. (FIG. 6B) Interferon γ degranulation by GPC2 CAR T cell constructs measured by ELISA across multiple cell lines with varying GPC2 expression at various E:T ratios. (FIG. 6C) Cytotoxicity and interferon γ release by D3V3 and D3V4 CAR T cells against DAOY medulloblastoma cell line. E:T ratio 10:1. (FIG. 6D) Cytotoxicity and interferon γ release by D3V3 and D3V4 CART cells against 7316-913 high grade glioma cell line. E:T ratio 5:1.

FIGS. 7A-C. D3V3 mRNA GPC2 CAR T cells shows greatest cytotoxicity in vivo in NB-1643 patient-derived xenograft (PDX) model. (FIG. 7A) Tumor growth over time in mice treated with IV delivery of D3V3 CAR compared to CD19 CAR control. Each line represents one mouse. (FIG. 7B) Tumor growth over time in mice treated with IV delivery of D3V4 CAR compared to CD19 CAR control. Each line represents one mouse. (FIG. 7C) Tumor growth over time of mice treated with intratumoral delivery of D3V3 and D3V4 CAR compared to CD19 CAR control (left), and Kaplan-Meier progression free survival (right).

FIG. 8. Schematic for CAR-T cell therapy and GPC2 RNA CAR construct design.

FIGS. 9A-C. Alignment of amino acid sequences of GPC2 single-chain variable fragments and expression of derived GPC2 CAR constructs. (FIG. 9A) Amino acid sequence alignment of GPC2 targeted single chain variable fragments (scFv) in variable heavy chain (VH)-linker-variable light chain (VL) orientation of GPC2.D4 (SEQ ID NO: 1) and GPC2.D3 (SEQ ID NO: 2). Complementarity-determining regions (CDR) are shown in gray. (FIG. 9B) Schematic of CAR T-cell constructs used for testing of 2 different scFv's in variable heavy chain-linker-variable light chain and variable light chain-linker-variable heavy chain orientation. (FIG. 9C) Expression of GPC2 CAR T-cell constructs on the surface of primary human T-cells assessed by the capacity to binding fluorescently labelled soluble, recombinant, human GPC2.

FIG. 10. GPC2 expression on Neuroblastoma cell lines. Cell surface expression of GPC2 on Neuroblastoma cell lines and CHO negative control stained with D3-IgG (labelled with Dylight650).

FIGS. 11A-D. Binder prioritization-based capacity of CAR T-cells for antigen exposure driven cytokine produce, killing and signs of low tonic signaling in the absence of antigen. (FIG. 11A) IFNy secretion of all constructs in response to tumor cells harboring overexpressed (Kelly-GPC2) and native GPC2 site density (NBSD) and (FIG. 11B) baseline IFNy secretion of CAR T-cells in the absence of antigen. (FIG. 11C) Killing capacity of GPC2 CAR T-cells against overexpressed (Kelly-GPC2) and native GPC2 site density (NBSD) at a 1:1 effector of tumor cell ratio. (FIG. 11D) Secretion of IL-2 of GPC2 CAR T-cells in response to overexpressed (Kelly-GPC2) and native GPC2 site density (NBSD).

FIGS. 12A-D. Engineered CAR constructs are ineffective against tumors expressing endogenous GPC2 antigen density. (FIG. 12A) Site density of GPC2 on overexpressed, engineered isogenic Kelly-GPC2 and endogenous GPC2 expressing neuroblastoma cell lines NBSD and SMS-SAN measured using Quantibrite beads. (FIG. 12B) IFNy secretion of GPC2 CAR constructs in response to overexpressed and endogenous GPC2 site density. (FIG. 12C) Capacity of GPC2 CAR T-cells to kill isogenic Kelly-GPC2 and (FIG. 12D) native GPC2 cell lines when challenged with 5× excess of tumor cells.

FIGS. 13A-E. CAR T-cells including a CH2CH3 spacer domain fail to improve GPC2 CAR functionality. (FIG. 13A) Schematic of GPC2 CAR constructs comprising an IgG4 derived CH2CH3 spacer domain. (FIG. 13B) Expression of D3VLVH.GPC2 long and short CAR T-cells assessed by staining with soluble, recombinant GPC2. (FIG. 13C) In vitro expansion of short and long GPC2.19 CAR T-cells shown as days post activation. (FIG. 13D) Killing capacity of short and long GPC2 CAR T-cells against neuroblastoma cell lines. (FIG. 13E) Cytokine production of short and long GPC2 CAR T-cells against neuroblastoma cell lines.

FIGS. 14A-B. GPC2 CAR T-cell constructs incorporating 28 transmembrane and signaling domains effectively target native GPC2 site density. (FIG. 14A) Cytokine production (IFNy to the left, IL-2 to the right) of GPC2.D3VLVH CAR T-cells compared to constructs incorporating CD28 hinge/transmembrane domains with either 41BBz or CD28 signaling domains. (FIG. 14B) Killing capacity of GPC2.D3VLVH CAR T-cells compared to constructs incorporating CD28 hinge/transmembrane domains with either 41BBz or CD28 signaling domains.

FIGS. 15A-F. D3 (M201)-based GPC2 DNA CAR T cells are potently cytotoxic to neuroblastoma preclinical models. (FIG. 15A) GPC2 CAR expression on T cells. D3 (M201) Long linker 28/28/41BB, D3 (M201)-based GPC2 CAR with CD28 based hinge/CD28 based Tm domain/41BB costimulatory domain and long linker; 28/28/28, D3(M201)-based GPC2 CAR with CD28 based hinge/CD28 based Tm domain/CD28 costimulatory domain and long linker. (FIG. 15B) Percent SYSY-GPC2 cell cytotoxicity of 8 different D3-based CAR constructs compared to UTD T cell control. (FIGS. 15C-D) Percent INFg (FIG. 15C) and CD107A (FIG. 15D) positive GPC2 CAR T cells utilizing 8 different D3 (M201)-based CAR constructs upon co-incubation with SYSY-GPC2 cells. (FIG. 15E) Neuroblastoma COG-N-421x patient-derived xenograft tumor growth after treatment with D3/M201-based GPC2 CAR T cells. (FIG. 15F) Mean weights of treatment cohorts of mice shown in FIG. 15E. UTD, untransduced T cells.

FIGS. 16A-B. Anti-tumor efficacy of D3 (M201)-VLVH-based CART cells in SMS-SAN metastatic xenograft model. (FIG. 16A) Study schema. (FIG. 16B) BLI data (correlating with tumor volume) of different D3 (M201)-VLVH-based in SMS-SAN metastatic model. *, p<0.05″; **, p<0.005; ***, p<0.0005; ****, p<0.00005.

### DESCRIPTION OF ILLUSTRATIVE EMBODIMENTS

The recent identification of glypican-2 (GPC2) as a cell surface oncoprotein in neuroblastoma, high grade glioma (HGG), and medulloblastoma provides the opportunity for the development of targeted immunotherapy. The inventors hypothesized that chimeric antigen receptor (CAR) T cell therapy directed against GPC2 could be achieved by either using in vitro transcribed RNA or by stably tranducing DNA constructs expressing GPC2 targeting CAR molecules.

The inventors created multiple CAR T cell constructs using the D3 and D4 GPC2 binders with manipulated heavy and light chain orientations. The resulting data show the utility of using either mRNA or DNA to efficiently design and test novel CAR T cells, providing a platform for clinical testing for proof of efficacy and screening for toxicity.

These and other aspects of the disclosure are described in greater detail below.

### I. GLYPICAN 2

Glypican-2 (GPC2) is a member of the six-member glypican family of heparan sulfate (HS) proteoglycans that are attached to the cell surface by a glycosylphosphatidylinositol (GPI) anchor and play diverse roles in growth factor signaling and cancer cell growth. GPC2 is also known as cerebroglycan proteoglycan and glypican proteoglycan 2. GPC2 genomic, mRNA and protein sequences are publicly available. In addition, human Glypican 2 mRNA and protein sequences can also be found in public databases, such as, for example, NCBI Gene ID 221914, Accession numbers NM_152742, and NP_689955, respectively, which are hereby incorporated by reference. The cell surface GPC2 protein has been shown to be expressed in the developing nervous system, participates in cell adhesion and is believed to regulate the growth and guidance of axons.

GPC2 has been recently identified as a cell surface protein several cancers, including pediatric cancers such as neuroblastoma, high grade glioma (HGG), medulloblastoma, and several other pediatric cancers and adult malignancies, which represents an opportunity for the development of new targeted immunotherapies. For example, in pediatric cancer, GPC2 has been shown to be expressed on neuroblastoma, retinoblastoma and medulloblastoma at comparable levels, while showing restricted normal tissue expression. Additionally, subsets of acute lymphoblastic leukemia, high-grade glioma and rhabdomyosarcoma express GPC2. GPC2 is also highly expressed on small cell lung cancer, a common and nearly universally lethal cancer. In addition, numerous adult malignancies could benefit from GPC2-targeted immunotherapeutics, as evaluating GPC2 expression in adult cancer utilizing data sourced from The Cancer Genome Atlas (TCGA). Due to this preferential expression, GPC2 represents a potential candidate for targeted immunotherapy. It is present on the cell surface of numerous childhood and adult malignancies and demonstrates high differential expression between tumor and normal tissues.

### II. PRODUCING MONOCLONAL ANTIBODIES

A. General Methods

Antibodies to Glypican 2 may be produced by standard methods as are well known in the art (see, e.g., Antibodies: A Laboratory Manual, Cold Spring Harbor Laboratory, 1988; U.S. Pat. No. 4,196,265). The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this disclosure were generated using the SP2/0/mIL-6 cell line, an IL-6 secreting derivative of the SP2/0 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.

The selected hybridomas are then serially diluted or single cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Single Chain/Single Domain Antibodies

A Single Chain Variable Fragment (scFv) is a fusion of the variable regions of the heavy and light chains of immunoglobulins, linked together with a short (usually serine, glycine) linker. This chimeric molecule, also known as a single domain antibody, retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single domain or single chain variable fragments lack the constant Fc region found in complete antibody molecules, and thus, the common binding sites (e.g., protein A/G) used to purify antibodies (single chain antibodies include the Fc region). These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Phage display can be used as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain.

Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stablizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug. U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

C. Chimeric Antigen Receptors and Nucleic Acid Sequences Coding Therefor

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of cancer-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signalling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain).

It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed. For example, chimeric CD28 and OX40 can be used with CD3-Zeta to transmit a proliferative/survival signal, or all three can be used together.

“First-generation” CARs typically had the intracellular domain from the CD3 ζ-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

Adoptive transfer of T cells expressing chimeric antigen receptors is a promising anti-cancer therapeutic as CAR-modified T cells can be engineered to target virtually any tumor associated antigen. There is great potential for this approach to improve patient-specific cancer therapy in a profound way. Following the collection of a patient's T cells, the cells are genetically engineered to express CARs specifically directed towards antigens on the patient's tumor cells, then infused back into the patient. Although adoptive transfer of CAR-modified T-cells is a unique and promising cancer therapeutic, there are significant safety concerns. Clinical trials of this therapy have revealed potential toxic effects of these CARs when healthy tissues express the same target antigens as the tumor cells, leading to outcomes similar to graft-versus-host disease (GVHD). A potential solution to this problem is engineering a suicide gene into the modified T cells. In this way, administration of a prodrug designed to activate the suicide gene during GVHD triggers apoptosis in the suicide gene-activated CAR T cells. This method has been used safely and effectively in hematopoietic stem cell transplantation (HSCT). Adoption of suicide gene therapy to the clinical application of CAR-modified T cell adoptive cell transfer has potential to alleviate GVHD while improving overall anti-tumor efficacy.

In some embodiments of the GPC2-targeting CAR disclosed herein, the VH sequence is operably linked downstream to the VL sequence. In some embodiments, the VH sequence is operably linked upstream to the VL sequence. As used herein, the term “upstream” in reference to an amino acid sequence refers to a location that is distal from a point of reference in an N-terminus to C-terminus direction of the amino acid sequence. Similarly, the term “downstream” refers to a location that is distal from a point of reference in a C-terminus to N-terminus direction of an amino acid sequence.

Generally, the transmembrane domain suitable for the GPC2-targeting CARs disclosed herein can be any one of the transmembrane domains known in the art. Non-limiting examples of suitable transmembrane domains include transmembrane domains derived from a CD28 transmembrane domain, a CD8a transmembrane domain, CTLA4 transmembrane domain, or a PD-I transmembrane domain. Accordingly, in some embodiments, the GPC2-targeting CAR of the disclosure includes a transmembrane domain derived from a CD28 transmembrane domain, a CD8a transmembrane domain, CTLA4 transmembrane domain, or a PD-I transmembrane domain. In some embodiments, the GPC2-targeting CAR includes a transmembrane domain derived from a CD28 transmembrane domain.

In some embodiments, the intracellular signaling domain of the GPC2-targeting CAR disclosed herein includes a co-stimulatory domain. Generally, the co-stimulatory domain suitable for the GPC2-targeting CARs disclosed herein can be any one of the co-stimulatory domains known in the art. Examples of suitable co-stimulatory domains include, but are not limited to, co-stimulatory polypeptide sequences derived from 4-IBB (CD137), CD27, CD28, OX40 (CD 134), and co-stimulatory inducible T-cell costimulatory (ICOS) polypeptide sequences. Accordingly, in some embodiments, the co-stimulatory domain of the GPC2-targeting CAR disclosed herein is selected from the group consisting of a co-stimulatory 4-IBB (CD137) polypeptide sequence, a co-stimulatory CD27 polypeptide sequence, a co-stimulatory CD28 polypeptide sequence, a co-stimulatory OX40 (CD134) polypeptide sequence, and a co-stimulatory inducible T-cell costimulatory (ICOS) polypeptide sequence. In some embodiments, the GPC2-targeting CAR includes a co-stimulatory domain derived from a co-stimulatory 4-1BB (CD137) polypeptide sequence. In some embodiments, the GPC2-targeting CAR includes a co-stimulatory domain derived from a co-stimulatory CD28 polypeptide sequence.

In some embodiments, the GPC2-targeting CAR further includes an extracellular hinge domain (e.g., hinge region) or “linker”. The term “hinge domain” generally refers to a flexible polypeptide connector region or “linker” disposed between the targeting moiety and the transmembrane domain. These sequences are generally derived from IgG subclasses (such as IgG 1 and IgG4), IgD and CD8 domains, of which IgG 1 has been most extensively used. In some embodiments, the hinge/linker domain provides structural flexibility to flanking polypeptide regions. The hinge/linker domain may consist of natural or synthetic polypeptides. It will be appreciated by those skilled in the art that hinge/linker domains may improve the function of the CAR by promoting optimal positioning of the antigen-binding moiety in relationship to the portion of the antigen recognized by the same. It will be appreciated that, in some embodiments, the hinge/linker domain may not be required for optimal CAR activity. In some embodiments, a beneficial hinge/linker domain comprising a short sequence of amino acids promotes CAR activity by facilitating antigen-binding by, e.g., relieving any steric constraints that may otherwise alter antibody binding kinetics. The sequence encoding the hinge/linker domain may be positioned between the antigen recognition moiety and the transmembrane domain. In some embodiments, the hinge/linker domain is operably linked downstream of the antigen-binding moiety and upstream of the transmembrane domain.

The hinge/linker sequence can be any moiety or sequence derived or obtained from any suitable molecule. For example, in some embodiments, the hinge/linker sequence can be derived from the human CD8a molecule or a CD28 molecule and any other receptors that provide a similar function in providing flexibility to flanking regions. The hinge/linker domain can have a length of from about 4 amino acid (aa) to about 50 aa, e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. Suitable hinge/linker domains can be readily selected and can be of any of a number of suitable lengths, uch as from 1 amino acid (e.g., Gly) to 20 aa, from 2 aa to 15 aa, from 3 aa to 12 aa, including 4 aa to 10 aa, 5 aa to 9 aa, 6 aa to 8 aa, or 7 aa to 8 aa, and can be 1, 2, 3, 4, 5, 6, or 7 aa.

The terms “long linker” and “short linker” are used throughout the application and are meant to refer to the following”

“long linker” amino acid sequence: GGGGSGGGGSGGGGS (SEQ ID NO: 4)

“short linker” amino acid sequence: GGGGS (SEQ ID NO: 41)

Non-limiting examples of suitable hinge/linker domains include a CD8 hinge domain, a CD28 hinge domain, a CTLA4 hinge domain, or an IgG4 hinge domain. In some embodiments, the hinge/linker domain can include regions derived from a human CD8a (a.k.a. CD8a) molecule or a CD28 molecule and any other receptors that provide a similar function in providing flexibility to flanking regions. In some embodiments, the GPC2-targeting CAR disclosed herein includes a hinge domain derived from a CD8a hinge domain.

In some embodiments, the GPC2-targeting CAR disclosed herein includes a hinge domain derived from a CD28 hinge domain.

In some embodiments, the CAR disclosed herein further includes an extracellular spacer domain including one or more intervening amino acid residues that are positioned between the anti-GPC2 scFV region and the extracellular hinge/linker domain. In some embodiments, the extracellular hinge/linker domain is operably linked downstream to the anti-GPC2 scFV region and upstream to the hinge/linker domain. In principle, there are no particular limitations to the length and/or amino acid composition of the extracellular spacer. In some embodiments, any arbitrary single-chain peptide comprising about one to about 300 amino acid residues (e.g., 2, 3, 4, 5, 6, 7, 8, 9, 10, 11, 12, 13, 14, 15, 16, 17, 18, 19, 20, etc. amino acid residues) can be used as an extracellular spacer. In some embodiments, the extracellular spacer includes about 5 to 50, about 10 to 60, about 20 to 70, about 30 to 80, about 40 to 90, about 50 to 100, about 60 to 120, about 70 to 150, about 100 to 200, about 150 to 250, about 200 to 300, about 30 to 60, about 20 to 80, about 30 to 90 amino acid residues. In some embodiments, the extracellular spacer includes about 1 to 10, about 50 to 100, about 100 to 150, about 150 to 200, about 200 to 300, about 20 to 80, about 40 to 120, about 200 to 250 amino acid residues. In some embodiments, the extracellular hinge/linker includes about 40 to 70, about 50 to 80, about 60 to 80, about 70 to 90, or about 80 to 100 amino acid residues. In some embodiments, the extracellular hingle/linker includes about 1 to 10, about 5 to 15, about 10 to 20, about 15 to 25 amino acid residues. In some embodiments, the extracellular hinge/linker includes about 220, 225, 230, 235, or 240 amino acid residues. In some embodiments, the extracellular hinge/linker includes 229 amino acid residues. In some embodiments, the length and amino acid composition of the extracellular hinge/linker can be optimized to vary the orientation and/or proximity of the anti-GPC2 scFV region and the extracellular hinge/linker domain to one another to achieve a desired activity of the GPC2-targeting CAR. In some embodiments, the orientation and/or proximity of the anti-GPC2 scFV region and the extracellular hinge/linker domain to one another can be varied and/or optimized as a “tuning” tool or effect that would enhance or reduce the efficacy of the GPC2 CAR. In some embodiments, the orientation and/or proximity of the anti-GPC2 scFV region and the extracellular hinge/linker domain to one another can be varied and/or optimized to create a partially functional or partially functional versions of the GPC2 CAR. In some embodiments, the extracellular hinge/linker domain includes an amino acid sequence corresponding to an IgG4 hinge domain and an IgG4 CH2-CH3 domain.

In some embodiments, the intracellular signaling domain of the GPC2-targeting CAR disclosed herein includes a CD3ζ intracellular signaling domain. In some embodiments of the disclosure, the GPC2-targeting CAR includes a) an anti-GPC2 scFv region; b) a CD28 hinge domain; c) a CD28 transmembrane domain; and d) an intracellular signaling domain including a co-stimulatory domain derived from a 4-1BBz co-stimulatory domain or a CD28 co-stimulatory domain.

In one aspect, some embodiments of the disclosure relate to a recombinant nucleic acid molecule including a nucleic acid sequence that encodes a GPC2-targeting CAR as disclosed herein, or an antibody as disclosed herein.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an anti sense strand). A nucleic acid molecule may contain unconventional or modified nucleotides. The terms “polynucleotide sequence” and “nucleic acid sequence” as used herein interchangeably refer to the sequence of a polynucleotide molecule.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the recombinant nucleic acid molecule is operably linked to a heterologous nucleic acid sequence, such as, for example a structural gene that encodes a protein of interest or a regulatory sequence (e.g., promoter sequence). In some embodiments, the recombinant nucleic acid molecule is further defined as an expression cassette or a vector. In some embodiments, the vector is a lentiviral vector, an adeno virus vector, an adeno-associated virus vector, or a retroviral vector.

Some embodiments disclosed herein relate to vectors or expression cassettes including a recombinant nucleic acid molecule as disclosed herein. As used herein, the term “expression cassette” refers to a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell and/or into a subject. As such, the term expression cassette may be used interchangeably with the term “expression construct.”

Chimeric antigen receptors (CARs) according to the present disclosure may be defined, in the first instance, by their binding specificity, which in this case is for Glypican 2. CARs may also be defined by the sequences disclosed herein, or may vary from the sequences provided above, optionally using methods discussed in greater detail below. For example, amino sequences may vary from those set out above in that (a) the variable regions may be segregated away from the constant domains of the light chains, (b) the amino acids may vary from those set out above while not drastically affecting the chemical properties of the residues thereby (so-called conservative substitutions), (c) the amino acids may vary from those set out above by a given percentage, e.g., 80%, 85%, 90%, 91%, 92%, 93%, 94%, 95%, 96%, 97%, 98% or 99% homology. Alternatively, the nucleic acids encoding the antibodies may (a) be segregated away from the constant domains of the light chains, (b) vary from those set out above while not changing the residues coded thereby, (c) may vary from those set out above by a given percentage, e.g., 70%, 75%, 80%, 85%, 90%, 91%, 92%, 93%, 94%, 95%, 96%, 97%, 98% or 99% homology, or (d) vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C.

In making conservative changes in amino acid sequence, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include arginine and lysine; glutamate and aspartate; serine and threonine; glutamine and asparagine; and valine, leucine and isoleucine.

D. Expression

Nucleic acids according to the present disclosure will encode CARs. As used in this application, the term “a nucleic acid encoding a Glypican 2 CAR” refers to a nucleic acid molecule that has been isolated free of total cellular nucleic acid. In certain embodiments, the disclosure concerns receptors that are encoded by any of the sequences set forth herein.

TABLE 2
CODONS

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments of the present disclosure include those encoding biologically functional equivalent proteins of the sequences described above. Such sequences may arise as a consequence of codon redundancy and amino acid functional equivalency that are known to occur naturally within nucleic acid sequences and the proteins thus encoded. Alternatively, functionally equivalent proteins may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous. The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DlA dopamine receptor gene (Lee, et al., 1997), insulin-like growth factor II (Wu et al., 1997), human platelet endothelial cell adhesion molecule-1 (Almendro et al., 1996).

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

2. IRES

In certain embodiments of the disclosure, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

5. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

8. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

9. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis,

Gaucher disease, renal disease and arthritis (Robbins and Ghivizzani, 1998; Imai et al., 1998; U.S. Pat. No. 5,670,488). Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

10. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

11. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to;

gpt, that confers resistance to mycophenolic acid; neo, that confers resistance to the aminoglycoside G418; and hygro, that confers resistance to hygromycin.

### III. PHARMACEUTICAL FORMULATIONS AND TREATMENT OF CANCER

A. Cancers

Cancer results from the outgrowth of a clonal population of cells from tissue. The development of cancer, referred to as carcinogenesis, can be modeled and characterized in a number of ways. An association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Cancer cells to which the methods of the present disclosure can be applied include generally any cell that expresses Glypican 2, and more particularly, that overexpresses Glypican 2. Cancer cells that may be treated according to the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, medulloblastoma, neuroblastoma, or leukemia.

In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present disclosure may be particularly applied to such cancers so as to render them resectable, to prolong or re-induce remission, to inhibit angiogenesis, to prevent or limit metastasis, and/or to treat multi-drug resistant cancers. At a cellular level, this may translate into killing cancer cells, inhibiting cancer cell growth, or otherwise reversing or reducing the malignant phenotype of tumor cells.

B. Formulation and Administration

The present disclosure provides pharmaceutical compositions comprising anti-Glypican 2 receptors and cells expressing the same. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The receptors, nucleic acids and cells of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or admininstration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

C. Combination Therapies

In the context of the present disclosure, it also is contemplated that anti-Glypican 2 CAR T-cells described herein could be used similarly in conjunction with chemo- or radiotherapeutic intervention, or other treatments. It also may prove effective, in particular, to combine anti-Glypican 2 CAR T-cells with other therapies that target different aspects of Glypican 2 function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with an anti-Glypican 2 CAR T-cells according to the present disclosure and at least one other agent. These compositions would be provided in a combined amount effective to kill or inhibit proliferation of the cell. This process may involve contacting the cells with the anti-Glypican 2 CAR T-cells according to the present disclosure and the other agent(s) or factor(s) at the same time. This may be achieved by contacting the cell with a single composition or pharmacological formulation that includes both agents, or by contacting the cell with two distinct compositions or formulations, at the same time, wherein one composition includes the anti-Glypican 2 CAR T-cells according to the present disclosure and the other includes the other agent.

Alternatively, the anti-Glypican 2 CAR T-cell therapy may precede or follow the other agent treatment by intervals ranging from minutes to weeks. In embodiments where the other agent and the anti-Glypican 2 CAR T-cells are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either anti-Glypican 2 CAR T-cells the other agent will be desired. Various combinations may be employed, where an anti-Glypican 2 CAR T cell according to the present disclosure is “A” and the other therapy is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. Again, to achieve cell killing, both agents are delivered to a cell in a combined amount effective to kill the cell. Agents or factors suitable for cancer therapy include any chemical compound or treatment method that induces damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition. A combination therapy may also include surgery. Various modes of these therapies are discussed below.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, y-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believed to be particularly efficacious in reducing the reoccurance of the tumor. Additionally, the compounds of the present disclosure can also be used in a neoadjuvant setting.

It also should be pointed out that any of the foregoing therapies may prove useful by themselves in treating cancer. The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. KITS

In still further embodiments, there are provided kits for use with the methods described above. The kits will thus comprise, in suitable container means, a CAR, a nucleic acid encoding a CAR, or a cell expressing a CAR first that binds to Glypican 2 antigen. The container means of the kits will generally include at least one vial, test tube, flask, bottle, syringe or other container means, into which the cell may be placed, or preferably, suitably aliquoted. The kits will also include a means for containing the CAR, nucleic acid or cell and any other reagent in close confinement for commercial sale. Such containers may include injection or blow-molded plastic containers into which the desired vials are retained.

### V. EXAMPLES

The following examples are included to demonstrate preferred embodiments. It should be appreciated by those of skill in the art that the techniques disclosed in the examples which follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

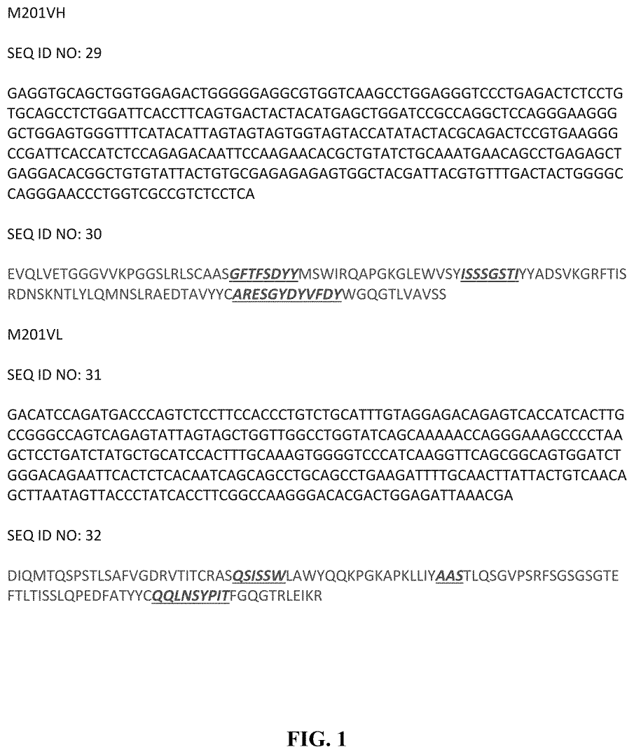

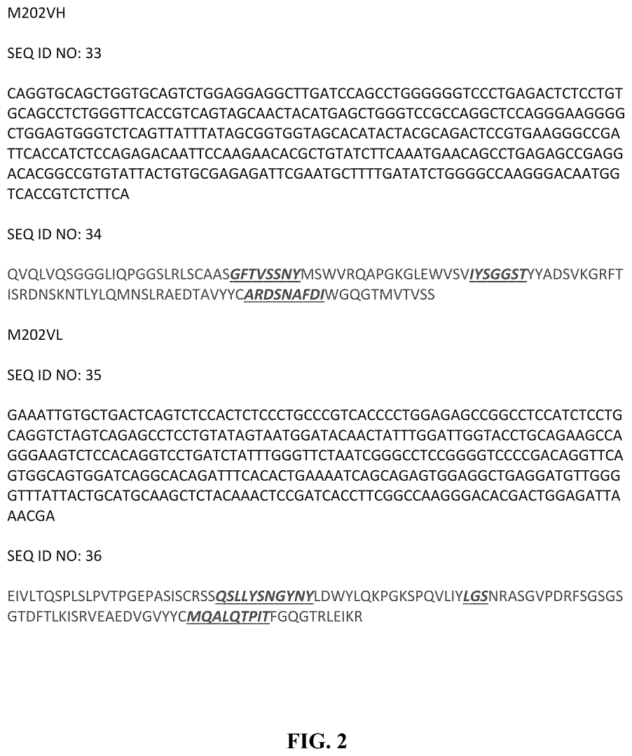

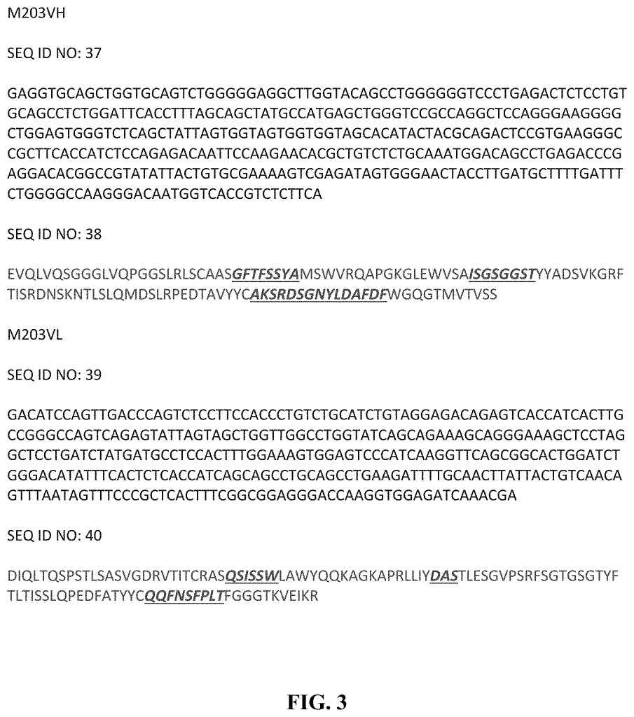

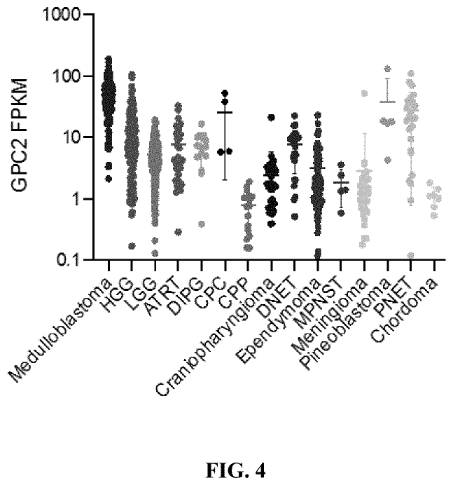

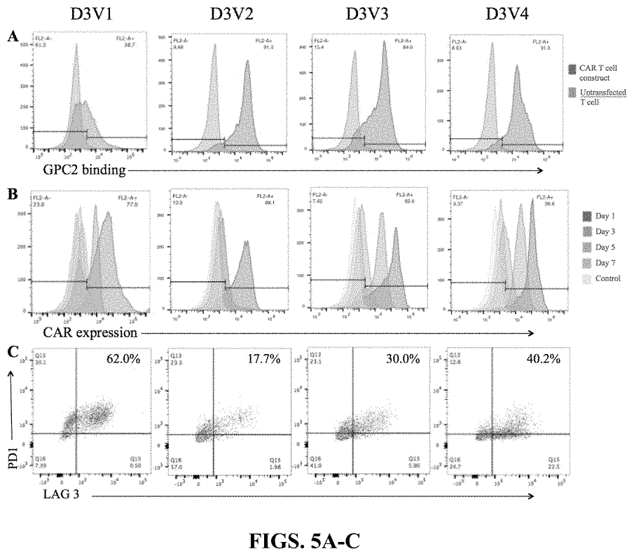

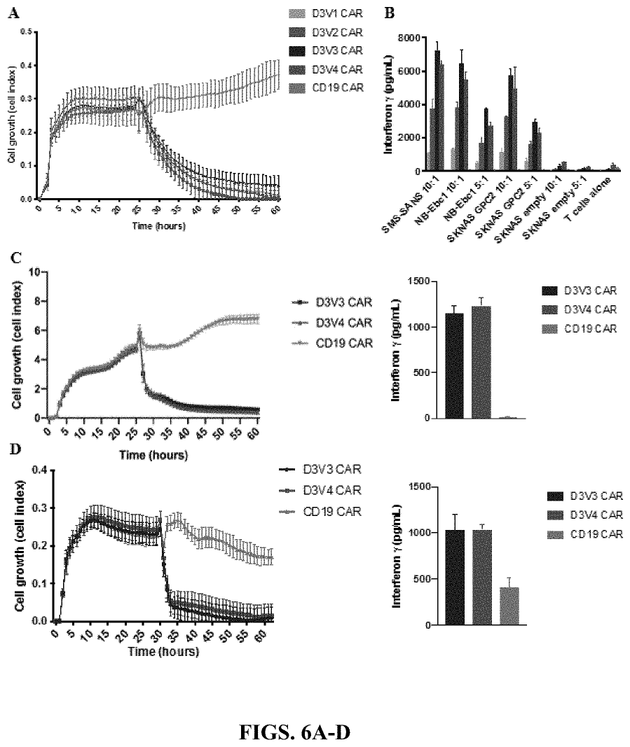

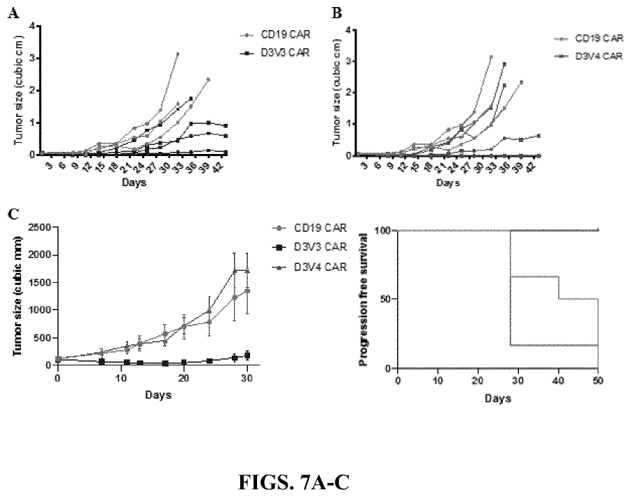

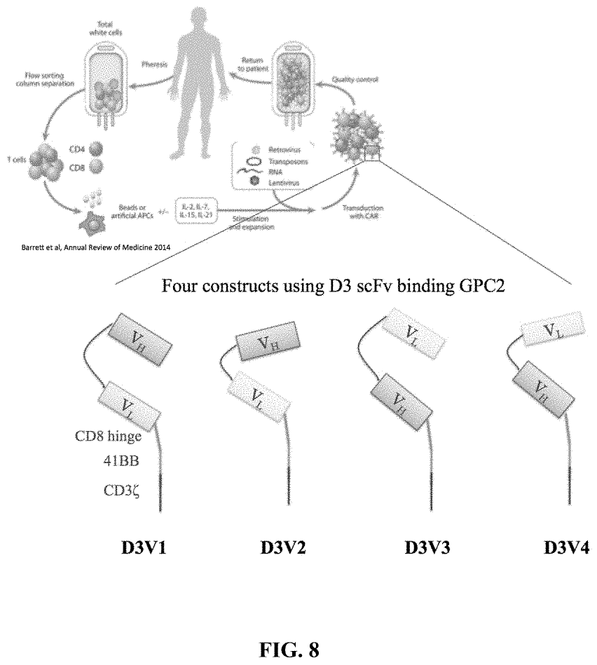

A panel of three fully human antibodies (m201, m202 and m203) specifically targeting cancer cell-associated GPC2 were isolated from a phage display antibody library and affinity matured. In vitro characterization demonstrates that these antibodies possess promising therapeutic activity for use in CAR-T, antibody drug conjugate (ADC) and bispecific antibody development for cancer therapy. The sequences of the antibodies are shown in FIGS. 1-3.

GPC2 was recently identified as novel oncogene and immunotherapeutic target in neuroblastoma and medulloblastoma. The inventors created mutliple different RNA CAR constructs using a GPC2-specific scFv paired with 4-1BB and CD3 co-stimulatory domains, with varied heavy and light chain orientation and linker length between chains. They evaluated CAR persistence, markers of T cell exhaustion, and cellular cytotoxicity against four primary and two isogenic neuroblastoma cell lines and three primary HGG cell lines. All four constructs showed >80% CAR expression and GPC2-specific binding by flow cytometry. CAR molecules in the light-to-heavy (VL-VH) configuration showed persistence on the surface over seven days and increased cytotoxicity compared to heavy-to-light (VH-VL) configuration. VH-VL configuration with long linker provided the weakest cytotoxic effect, and evaluation of negative checkpoint regulators revealed the highest expression of PD1 and Lag3 (62% vs. 17-40% in other constructs, p<0.0001). Based on the in vitro data, the two VL-VH CAR constructs were chosen for testing in murine flank models of neuroblastoma treated with IV GPC2 CAR T cells once weekly for three doses. At day 14, animals treated with both VL-VH CAR constructs had reduced tumor burden compared to CD19 CAR controls (p<0.01), with several animals showing complete response. Studies are currently underway evaluating efficacy in orthotopic models of pediatric HGG using local delivery.

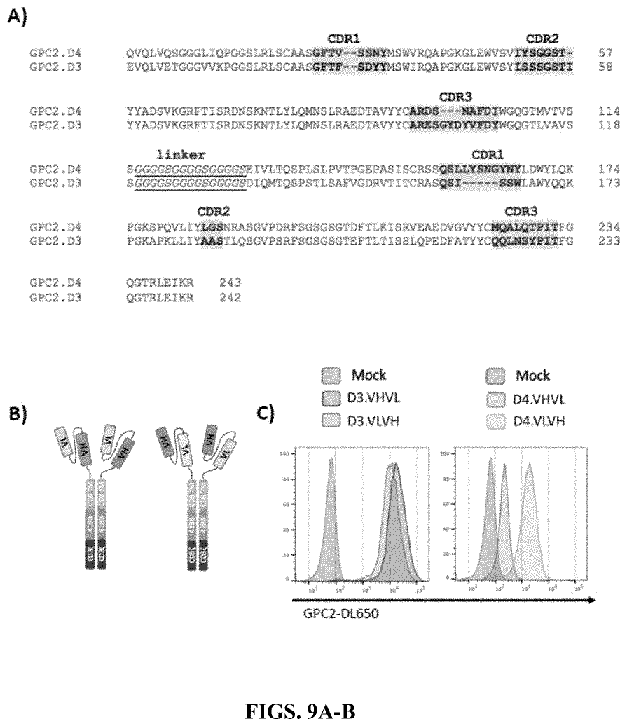

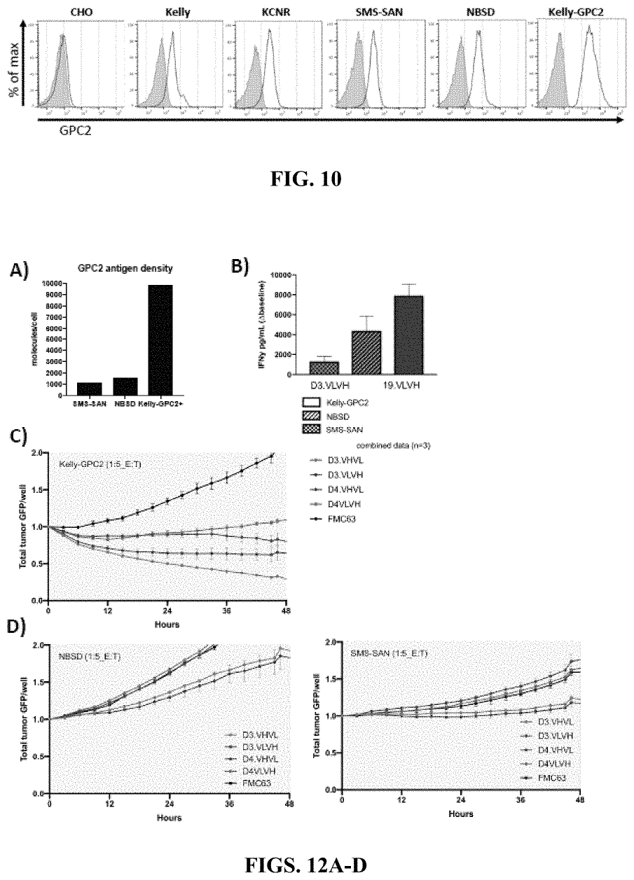

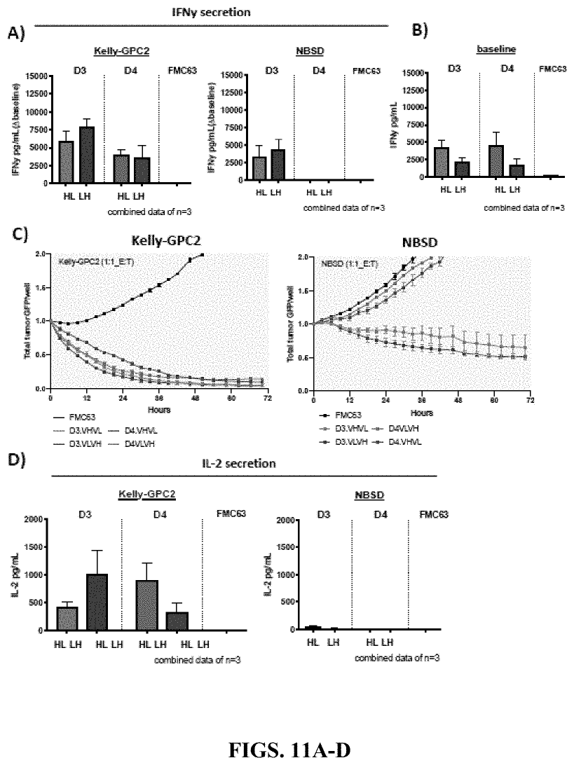

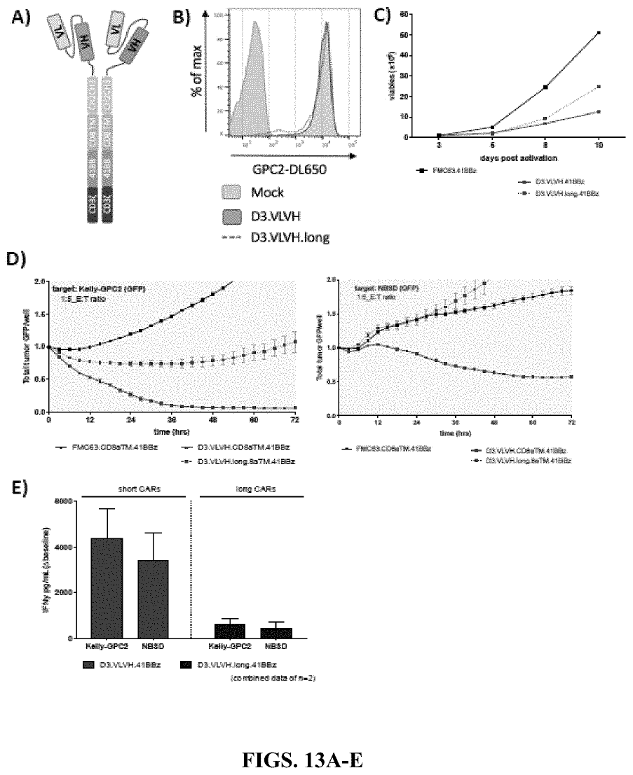

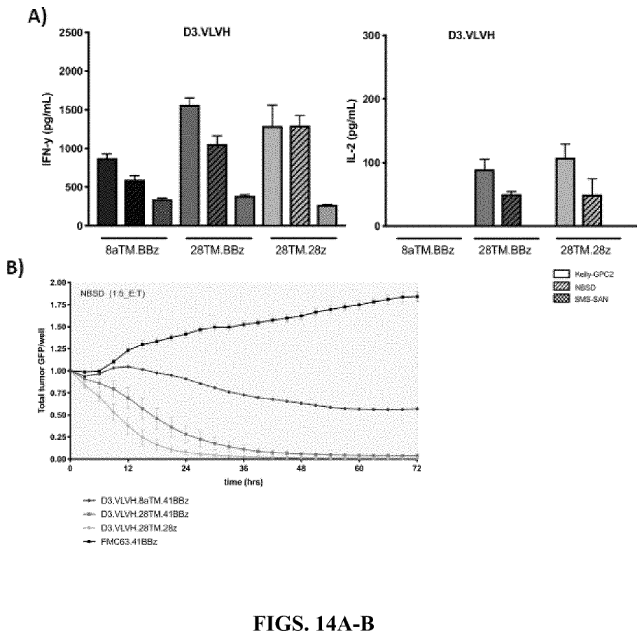

Stable expression of multiple CAR T-cell constructs was accomplished by engineering DNA-based second-generation CAR vectors based upon 2 of the reported GPC2 scFvs (D3 and D4) and following retroviral transduction in primary human T-cells. Initial constructs engineered possessing a CD8a hinge and transmembrane domain and a 41BBz signaling domain, in two orientations with either N-terminal variable heavy chain or N-terminal variable light chain (FIG. 9B) showed stable cell surface expression and bound soluble, recombinant GPC2 (FIG. 9C). These constructs showed potent in vitro efficacy and cytokine production (IFNy, IL2) against isogenic target cells engineered to express GPC2 at levels comparable to in vivo levels of GPC2 (Kelly-GPC2) at 1:1 effector to target ratios (FIGS. 11A-D). Furthermore, the inventors demonstrated that incorporating CD28-H/TM and co-stimulatory domains into these CAR constructs exhibit additional CAR T cell potency advantages when targeting GPC2-epressing tumors (FIGS. 14A-B). Taken together, these data show that utilizing DNA-based CAR vectors and viral transduction, stable CAR T cells targeting GPC2 can be engineered that enact potent killing effects on GPC2-expressing cancer cells.

These data show that mRNA offers a quick and iterative method to test novel CAR T cells, and that GPC2 is a promising CAR T cell target in neuroblastoma, medulloblastoma, as well as a subset of high-grade gliomas and other pediatric malignant brain tumors. RNA GPC2 CAR T cells in the light to heavy D3 scFv chain with long linker configuration provided strongest cytotoxic effect with no evidence of toxicity in murine models.

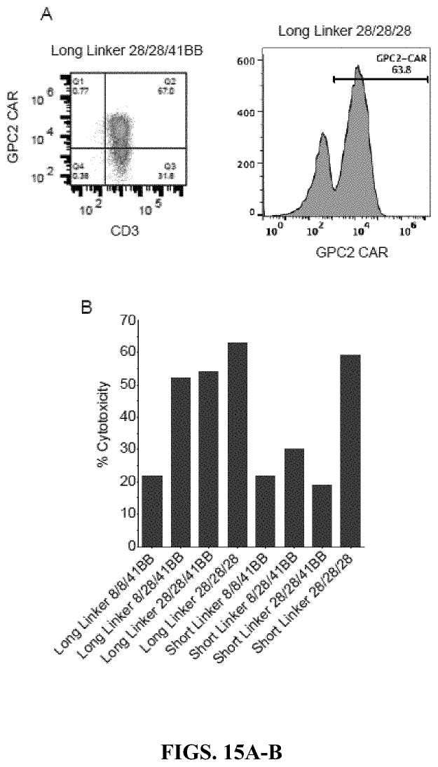

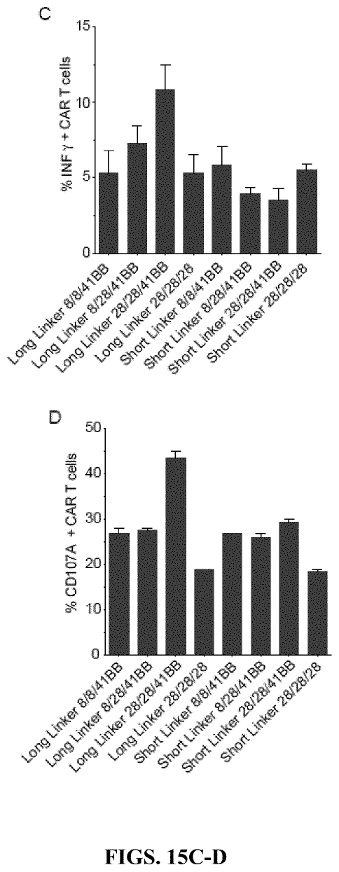

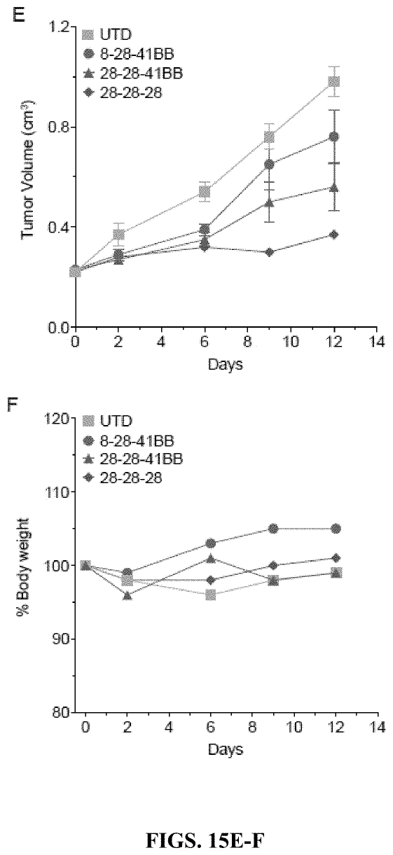

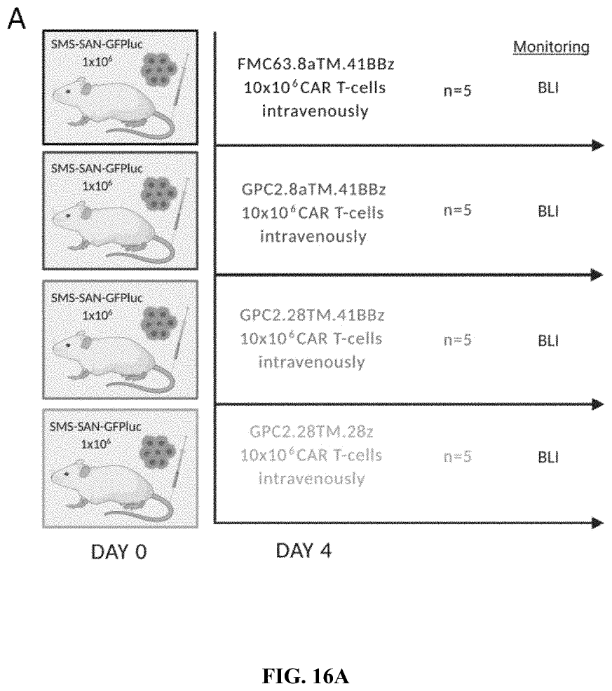

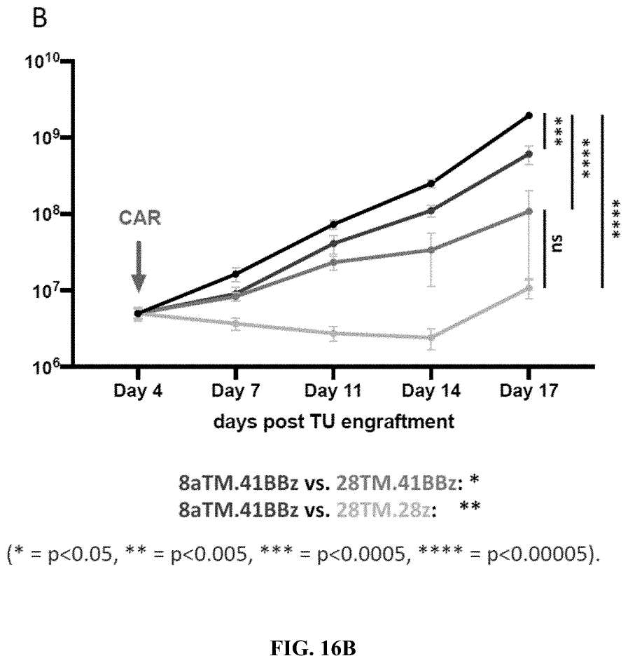

D3 (M201)-based GPC2 DNA CAR T cells transduced via lentiviruses (FIGS. 15A-F) and retroviruses (FIGS. 16A-B) are also potently cytotoxic to neuroblastoma preclinical models. GPC2 CARs are robustly expressed on T cells (FIG. 15A) and are cytotoxic to isogenic SYSY-GPC2 neruoblastoma cells (FIG. 15B), with co-culture resulting in concurrent T cell activation with increased INFy and CD107A T cell expression (FIGS. 15C-D). D3 (M201) long linker 28/28/41BB (D3 (M201)-based GPC2 CARs with CD28 based hinge/CD28 based Tm/41BB costimulatory domains) and long linker 28/28/28 (D3 (M201)-based GPC2 CAR with CD28 based hinge/CD28 based Tm/CD28 costimulatory domains) showed potent in vivo activity inducing robust COG-N-421x neuroblastoma patient-derived xenograft tumor regression and was very well-tolerated (FIGS. 15E-F). D3 (M201)-based GPC2 CAR T cells also induced tumor regression in a metastatic SMS-SAN neuroblastoma model (FIGS. 16A-B)

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,683,202
U.S. Pat. No. 4,684,611
U.S. Pat. No. 4,816,567
U.S. Pat. No. 4,867,973
U.S. Pat. No. 4,879,236
U.S. Pat. No. 4,938,948
U.S. Pat. No. 4,952,500
U.S. Pat. No. 5,021,236
U.S. Pat. No. 5,141,648
U.S. Pat. No. 5,196,066
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,563,250
U.S. Pat. No. 5,565,332
U.S. Pat. No. 5,580,859
U.S. Pat. No. 5,589,466
U.S. Pat. No. 5,610,042
U.S. Pat. No. 5,656,610
U.S. Pat. No. 5,670,488
U.S. Pat. No. 5,702,932
U.S. Pat. No. 5,736,524
U.S. Pat. No. 5,780,448
U.S. Pat. No. 5,789,215
U.S. Pat. No. 5,824,544
U.S. Pat. No. 5,856,456
U.S. Pat. No. 5,858,744
U.S. Pat. No. 5,871,982
U.S. Pat. No. 5,871,986
U.S. Pat. No. 5,880,270
U.S. Pat. No. 5,925,565
U.S. Pat. No. 5,928,906
U.S. Pat. No. 5,935,819
U.S. Pat. No. 5,945,100
U.S. Pat. No. 5,981,274
U.S. Pat. No. 5,994,624
“Antibodies: A Laboratory Manual,” Cold Spring Harbor Press, Cold Spring Harbor, NY, 1988.
Abbondanzo et al., Am. J. Pediatr. Hematol. Oncol., 12(4), 480-489, 1990.
Allred et al., Arch. Surg., 125(1), 107-113, 1990.
Almendro et al., J. Immunol., 157(12):5411-5421, 1996.
Atherton et al., Biol. of Reproduction, 32, 155-171, 1985.
Ausubel et al., Current Protocols in Molecular Biology, Greene Publishing Associates and Wiley Interscience, N.Y., 1994.
Beidler et al., J. Immunol., 141(11):4053-4060, 1988.
Bosse et al., Cancer Cell, 32:295-309, e212, 2017
Brown et al., J. Immunol. Meth., 12;130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Carbonelli et al., FEMS Microbiol. Lett., 177(1):75-82, 1999.
Chandler et al., Proc. Natl. Acad. Sci. USA, 94(8):3596-601, 1997.
Chen and Okayama, Mol. Cell Biol., 7(8):2745-2752, 1987.
Cocea, Biotechniques, 23(5):814-816, 1997.
Coffey et al., Science, 282(5392):1332-1334, 1998.
De Jager et al., Semin. Nucl. Med. 23(2), 165-179, 1993.
Dholakia et al., J. Biol. Chem., 264, 20638-20642, 1989.
Doolittle and Ben-Zeev, Methods Mol. Biol., 109, 215-237, 1999.
EP Application 125,023
EP Application 171,496
EP Application 173,494
EP Application 184,187
EP Application 273,085
Fechheimer et al., Proc Natl. Acad. Sci. USA, 84:8463-8467, 1987.
Fraley et al., Proc. Natl. Acad. Sci. USA, 76:3348-3352, 1979.
Gefter et al., Somatic Cell Genet., 3:231-236, 1977.
Gnant et al., Cancer Res., 59(14):3396-403, 1999.
Gnant et al., J. Natl. Cancer Inst., 91(20):1744-1750, 1999.
Goding, In: Monoclonal Antibodies: Principles and Practice, 2d ed., Orlando, Fla., Academic Press, 60-61, 65-66, 71-74, 1986.
Gopal, Mol. Cell Biol., 5:1188-1190, 1985.
Graham and Van Der Eb, Virology, 52:456-467, 1973.
Greene et al., Immunology Today, 10:272, 1989.
Gulbis and Galand, Hum. Pathol. 24(12), 1271-1285, 1993.
Harland and Weintraub, J. Cell Biol., 101(3):1094-1099, 1985.
Imai et al., Nephrologie, 19(7):397-402, 1998.
Jones et al., Nature, 321:522-525, 1986.
Kaeppler et al., Plant Cell Rep., 8:415-418, 1990.
Kaneda et al., Science, 243:375-378, 1989.
Kato etal, J. Biol. Chem., 266:3361-3364, 1991.
Khatoon et al., Ann. of Neurology, 26, 210-219, 1989.
King et al., J. Biol. Chem., 269, 10210-10218, 1989.
Kohler and Milstein, Eur. J. Immuno!., 6, 511-519, 1976.
Kohler and Milstein, Nature, 256, 495-497, 1975.
Kraus et al. FEBS Lett., 428(3):165-170, 1998.
Kyte and Doolittle, J. Mol. Biol., 157(1):105-132, 1982.
Lareyre et al., J. Biol. Chem., 274(12):8282-8290, 1999.
Levenson et al., Hum. Gene Ther., 9(8):1233-1236, 1998.
Lundstrom, J. Recept Signal Transduct. Res., 19(1-4):673-686, 1999.
Macejak and Sarnow, Nature, 353:90-94, 1991.
Morrison, Science, 229(4719):1202-1207, 1985.
Nakamura et al., In: Enzyme Immunoassays: Heterogeneous and Homogeneous Systems, Chapter 27, 1987.
Neumann et al., Proc. Natl. Acad. Sci. USA, 96(16):9345-9350, 1999.
Nicolau and Sene, Biochim. Biophys. Acta, 721:185-190, 1982.
Nicolau et al., Methods Enzymol., 149:157-176, 1987.
Nomoto et al., Gene, 236(2):259-271, 1999.
Omirulleh et al., Plant Mol. Biol., 21(3):415-428, 1993.
O'Shannessy et al., J. Immun. Meth., 99, 153-161, 1987.
Owens and Haley, J. Biol. Chem., 259, 14843-14848, 1987.
PCT Application PCT/US86/02269
PCT Application WO 86/01533
PCT Appin. WO 94/09699
PCT Appin. WO 95/06128
Pelletier and Sonenberg, Nature, 334(6180):320-325, 1988.
Posner et al., Hybridoma 6, 611-625, 1987.
Potrykus et al., Mol. Gen. Genet., 199(2):169-177, 1985.
Potter and Haley, Meth. Enzymol., 91, 613-633, 1983.
Remington's Pharmaceutical Sciences, 15^{th} Ed., 33:624-652, 1990.
Rippe, et al., Mol. Cell Biol., 10:689-695, 1990.
Robbins and Ghivizzani, Pharmacol Ther, 80(1):35-47, 1998.
Robbins et al., Trends Biotechnol., 16(1):35-40, 1998.
Sambrook et al., In: Molecular cloning: a laboratory manual, 2^{nd} Ed., Cold Spring Harbor Laboratory Press, Cold Spring Harbor, NY, 1989.
Shaw et al., J. Natl. Cancer Inst., 80(19):1553-1559, 1988.
Sun et al., J. Steroid Biochem., 26(1):83-92, 1987.
Tsumaki et al., J. Biol. Chem., 273(36):22861-2286
Verhoeyen et al., Science, 239(4847):1534-1536, 1988.
Wawrzynczak & Thorpe, Cancer Treat Res., 37:239-51, 1988.
Wong et al., Gene, 10:87-94, 1980.
Wood et al., J. Clin. Lab. Immunol., 17(4):167-171, 1985.
Wu et al., Biochem. Biophys. Res. Commun., 233(1):221-226, 1997.
Zhao-Emonet et al., Biochim. Biophys. Acta, 1442(2-3):109-119, 1998.

1. An isolated nucleic acid molecule encoding a chimeric antigen receptor (CAR),
wherein the CAR comprises an antigen binding domain, a flexible hinge domain, a transmembrane domain, a costimulatory signaling region, and an intracellular signaling domain, and
wherein the antigen binding domain binds selectively to a cancer cell-associated Glypican 2 (GPC2).

2. The isolated nucleic acid molecule of claim 1, wherein the antigen binding domain comprises an antibody or an antigen-binding fragment thereof.

3. The isolated nucleic acid molecule of claim 2, wherein the antigen-binding fragment is a Fab, a single-chain variable fragment (scFv), or a single-domain antibody.

4. The isolated nucleic acid molecule of claim 1, wherein the encoded antigen binding domain comprises:
(a) a heavy chain variable domain comprising the amino acid sequence of SEQ ID NO: 30 and a light chain variable domain comprising the amino acid sequence of SEQ ID NO: 32; or
(b) a heavy chain variable domain comprising the amino acid sequence of SEQ ID NO: 34, and a light chain variable domain comprising the amino acid sequence of SEQ ID NO: 36, or
(c) a heavy chain variable domain comprising the amino acid sequence of SEQ ID NO: 38 and a light chain variable domain comprising the amino acid sequence of SEQ ID NO: 40.

5. The isolated nucleic acid molecule of claim 1, wherein the encoded antigen binding domain comprises:
(a) a heavy chain variable domain including a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, and a light chain variable domain including a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; or
(b) a heavy chain variable domain including a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, and a light chain variable domain including a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; or
(c) a heavy chain variable domain including a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, and a light chain variable domain including a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

6. The isolated nucleic acid molecule of claim 2, wherein:
(a) the encoded antigen binding domain comprises a heavy chain variable domain comprising the amino acid sequence of SEQ ID NO: 30 and a light chain variable domain comprising the amino acid sequence of SEQ ID NO: 32; and
(b) the C-terminus of the light chain variable domain is fused to the N-terminus of a heavy chain variable domain by a flexible linker.

7. The isolated nucleic acid molecule of claim 6, wherein the linker is a peptide linker.

8. The isolated nucleic acid molecule of claim 7, wherein the peptide linker is at least 15 amino acids in length.

9. The isolated nucleic acid molecule of claim 8, wherein the peptide linker is a glycine-serine linker.

10. The isolated nucleic acid molecule of claim 1 wherein:
(a) the flexible hinge domain is from CD8α, CD28, or an immunoglobulin (Ig),
(b) the transmembrane domain comprises CD28 transmembrane domain,
(c) the costimulatory signaling region comprises a domain from CD28 , 41BB (CD137), OX40, or ICOS, and
(d) the intracellular signaling domain comprises a CD3-zeta domain or a high affinity FcεRI.

11. A chimeric antigen receptor (CAR) polypeptide, wherein:
(a) the CAR comprises an antigen binding domain, a flexible hinge domain, a transmembrane domain, a costimulatory signaling region, and an intracellular signaling domain; and
(b) the antigen binding domain binds selectively to cancer cell-associated Glypican 2 (GPC2).

12. The chimeric antigen receptor polypeptide of claim 11, wherein the antigen-binding fragment is a Fab, a single-chain variable fragment (scFv), or a single-domain antibody.

13. The chimeric antigen receptor (CAR) polypeptide of claim 11, wherein the encoded antigen binding domain comprises:
(a) a heavy chain variable domain comprising the amino acid sequence of SEQ ID NO: 30 and a light chain variable domain comprising the amino acid sequence of SEQ ID NO: 32; or
(b) a heavy chain variable domain comprising the amino acid sequence of SEQ ID NO: 34, and a light chain variable domain comprising the amino acid sequence of SEQ ID NO: 36, or
(c) a heavy chain variable domain comprising the amino acid sequence of SEQ ID NO: 38 and a light chain variable domain comprising the amino acid sequence of SEQ ID NO: 40.

14. The chimeric antigen receptor (CAR) polypeptide of claim 11, wherein the encoded antigen binding domain comprises:
(a) a heavy chain variable domain including a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, and a light chain variable domain including a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; or
(b) a heavy chain variable domain including a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, and a light chain variable domain including a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; or
(c) a heavy chain variable domain including a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, and a light chain variable domain including a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

15. The chimeric antigen receptor polypeptide of claim 13, wherein:
(a) the encoded antigen binding domain comprises a heavy chain variable domain comprising the amino acid sequence of SEQ ID NO: 30 and a light chain variable domain comprising the amino acid sequence of SEQ ID NO: 32; and
(b) the C-terminus of the light chain variable domain is fused to the N-terminus of a heavy chain variable domain by a flexible linker.

16. A genetically modified T cell comprising a nucleic acid sequence encoding a chimeric antigen receptor (CAR), or a genetically modified T cell comprising the isolated nucleic acid molecule of claim 1.

17. A genetically modified T cell comprising the chimeric antigen receptor of claim 11.

18. A method of making a genetically modified T cell comprising transducing the immune effector cell with the chimeric antigen receptor of claim 11.

19. A method of providing an anti-tumor immunity in a mammal, comprising administering to the mammal an effective amount of a population of genetically modified T cells of claim 16.

20. A method of treating a mammal having a disease associated with overexpression of a GPC2, the method comprising administering to the mammal an effective amount of a population of a genetically modified T cells of claim 16.

21. The genetically modified T cell of claim 16, wherein:
(a) the CAR induces interferon γ and Interleukin-2 secretion, and
(b) the genetically modified T cell exhibits cytotoxicity toward a GPC2 expressing cancer when the genetically modified T cell is exposed to the cancer cell-associated GPC2.

22. The method of claim 20, wherein the GPC2 expressing cancer is selected from the group consisting of sarcoma cell, a rhabdoid cancer cell, a neuroblastoma cell, retinoblastoma cell, or a medulloblastoma cell, uterine carcinosarcoma (UCS), brain lower grade glioma (LGG), thymoma (THYM), testicular germ cell tumors (TGCT), glioblastoma multiforme (GBM) and skin cutaneous melanoma (SKCM), liver hepatocellular carcinoma (LIHC), uveal melanoma (UVM), kidney chromophobe (KICH), thyroid cancer (THCA), kidney renal clear cell carcinoma (KIRC), kidney renal papillary cell carcinoma (KIRP), stomach adenocarcinoma (STAD), cholangiocarcinoma (CHOL), adenoid cystic carcinoma (ACC), prostate adenocarcinoma (PRAD), pheochromocytoma and paraganglioma (PCPG), DLBC, lung adenocarcinoma (LUAD), head-neck squamous cell carcinoma (HNSC), pancreatic adenocarcinoma (PAAD), breast cancer (BRCA), mesothelioma (MESO), colon and rectal adenocarcinoma (COAD). rectum adenocarcinoma (READ), esophageal carcinoma (ESCA), ovarian cancer (OV), lung squamous cell carcinoma (LUSC), bladder urothelial carcinoma (BLCA), sarcoma (SARC), or uterine corpus endometrial carcinoma (UCEC).


SEQUENCE LISTING (41 sequences)

SEQ ID NO: 1 - protein, 243 aa, Artificial Sequence
  Synthetic peptide
QVQLVQSGGGLIQPGGSLRLSCAASGFTVSSNYMSWVRQAPGKGLEWVSVIYSGGSTYYADSVKGRFTISRDNSKNTLYLQMNSLRAEDTAVYYCARDSNAFDIWGQGTMVTVSSGGGGSGGGGSGGGGSEIVLTQSPLSLPVTPGEPASISCRSSQSLLYSNGYNYLDWYLQKPGKSPQVLIYLGSNRASGVPDRFSGSGSGTDFTLKISRVEAEDVGVYYCMQALQTPITFGQGTRLEIKR

SEQ ID NO: 2 - protein, 242 aa, Artificial Sequence
  Synthetic peptide
EVQLVETGGGVVKPGGSLRLSCAASGFTFSDYYMSWIRQAPGKGLEWVSYISSSGSTIYYADSVKGRFTISRDNSKNTLYLQMNSLRAEDTAVYYCARESGYDYVFDYWGQGTLVAVSSGGGGSGGGGSGGGGSDIQMTQSPSTLSAFVGDRVTITCRASQSISSWLAWYQQKPGKAPKLLIYAASTLQSGVPSRFSGSGSGTEFTLTISSLQPEDFATYYCQQLNSYPITFGQGTRLEIKR

SEQ ID NO: 3 - protein, 245 aa, Artificial Sequence
  Synthetic peptide
EVQLVQSGGGLVQPGGSLRLSCAASGFTFSSYAMSWVRQAPGKGLEWVSAISGSGGSTYYADSVKGRFTISRDNSKNTLSLQMDSLRPEDTAVYYCAKSRDSGNYLDAFDFWGQGTMVTVSSGGGGSGGGGSGGGGSDIQLTQSPSTLSASVGDRVTITCRASQSISSWLAWYQQKAGKAPRLLIYDASTLESGVPSRFSGTGSGTYFTLTISSLQPEDFATYYCQQFNSFPLTFGGGTKVEIKR

SEQ ID NO: 4 - protein, 15 aa, Artificial Sequence
  Synthetic peptide
GGGGSGGGGSGGGGS

SEQ ID NO: 5 - protein, 115 aa, Artificial Sequence
  Synthetic peptide
QVQLVQSGGGLIQPGGSLRLSCAASGFTVSSNYMSWVRQAPGKGLEWVSVIYSGGSTYYADSVKGRFTISRDNSKNTLYLQMNSLRAEDTAVYYCARDSNAFDIWGQGTMVTVSS

SEQ ID NO: 6 - protein, 119 aa, Artificial Sequence
  Synthetic peptide
EIVLTQSPLSLPVTPGEPASISCRSSQSLLYSNGYNYLDWYLQKPGKSPQVLIYLGSNRASGVPDRFSGSGSGTDFTLKISRVEAEDVGVYYCMQALQTPITFGQGTRLEQGTRLEIKR

SEQ ID NO: 7 - protein, 119 aa, Artificial Sequence
  Synthetic peptide
EVQLVETGGGVVKPGGSLRLSCAASGFTFSDYYMSWIRQAPGKGLEWVSYISSSGSTIYYADSVKGRFTISRDNSKNTLYLQMNSLRAEDTAVYYCARESGYDYVFDYWGQGTLVAVSS

SEQ ID NO: 8 - protein, 108 aa, Artificial Sequence
  Synthetic peptide
DIQMTQSPSTLSAFVGDRVTITCRASQSISSWLAWYQQKPGKAPKLLIYAASTLQSGVPSRFSGSGSGTEFTLTISSLQPEDFATYYCQQLNSYPITFGQGTRLEIKR

SEQ ID NO: 9 - protein, 122 aa, Artificial Sequence
  Synthetic peptide
EVQLVQSGGGLVQPGGSLRLSCAASGFTFSSYAMSWVRQAPGKGLEWVSAISGSGGSTYYADSVKGRFTISRDNSKNTLSLQMDSLRPEDTAVYYCAKSRDSGNYLDAFDFWGQGTMVTVSS

SEQ ID NO: 10 - protein, 108 aa, Artificial Sequence
  Synthetic peptide
DIQLTQSPSTLSASVGDRVTITCRASQSISSWLAWYQQKAGKAPRLLIYDASTLESGVPSRFSGTGSGTYFTLTISSLQPEDFATYYCQQFNSFPLTFGGGTKVEIKR

SEQ ID NO: 11 - protein, 8 aa, Artificial Sequence
  Synthetic peptide
GFTVSSNY

SEQ ID NO: 12 - protein, 7 aa, Artificial Sequence
  Synthetic peptide
IYSGGST

SEQ ID NO: 13 - protein, 9 aa, Artificial Sequence
  Synthetic peptide
ARDSNAFDI

SEQ ID NO: 14 - protein, 11 aa, Artificial Sequence
  Synthetic peptide
QSLLYSNGYNY

SEQ ID NO: 15 - protein, 3 aa, Artificial Sequence
  Synthetic peptide
LGS

SEQ ID NO: 16 - protein, 9 aa, Artificial Sequence
  Synthetic peptide
MQALQTPIT

SEQ ID NO: 17 - protein, 8 aa, Artificial Sequence
  Synthetic peptide
GFTFSDYY

SEQ ID NO: 18 - protein, 8 aa, Artificial Sequence
  Synthetic peptide
ISSSGSTI

SEQ ID NO: 19 - protein, 12 aa, Artificial Sequence
  Synthetic peptide
ARESGYDYVFDY

SEQ ID NO: 20 - protein, 6 aa, Artificial Sequence
  Synthetic peptide
QSISSW

SEQ ID NO: 21 - protein, 3 aa, Artificial Sequence
  Synthetic peptide
AAS

SEQ ID NO: 22 - protein, 9 aa, Artificial Sequence
  Synthetic peptide
QQLNSYPIT

SEQ ID NO: 23 - protein, 8 aa, Artificial Sequence
  Synthetic peptide
GFTFSSYA

SEQ ID NO: 24 - protein, 8 aa, Artificial Sequence
  Synthetic peptide
ISGSGGST

SEQ ID NO: 25 - protein, 16 aa, Artificial Sequence
  Synthetic peptide
AKSRDSGNYLDADFDF

SEQ ID NO: 26 - protein, 6 aa, Artificial Sequence
  Synthetic peptide
QSISSW

SEQ ID NO: 27 - protein, 3 aa, Artificial Sequence
  Synthetic peptide
DAS

SEQ ID NO: 28 - protein, 9 aa, Artificial Sequence
  Synthetic peptide
QQFNSFPLT

SEQ ID NO: 29 - DNA, 357 bp, Artificial Sequence
  Synthetic primer
gaggtgcagctggtggagactgggggaggcgtggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactccgtgaagggccgattcaccatctccagagacaattccaagaacacgctgtatctgcaaatgaacagcctgagagctgaggacacggctgtgtattactgtgcgagagagagtggctacgattacgtgtttgactactggggccagggaaccctggtcgccgtctcctca

SEQ ID NO: 30 - protein, 119 aa, Artificial Sequence; identical to SEQ ID NO: 7
  Synthetic peptide

SEQ ID NO: 31 - DNA, 324 bp, Artificial Sequence
  Synthetic primer
gacatccagatgacccagtctccttccaccctgtctgcatttgtaggagacagagtcaccatcacttgccgggccagtcagagtattagtagctggttggcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctatcaccttcggccaagggacacgactggagattaaacga

SEQ ID NO: 32 - protein, 108 aa, Artificial Sequence; identical to SEQ ID NO: 8
  Synthetic peptide

SEQ ID NO: 33 - DNA, 345 bp, Artificial Sequence
  Synthetic primer
caggtgcagctggtgcagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagattcgaatgcttttgatatctggggccaagggacaatggtcaccgtctcttca

SEQ ID NO: 34 - protein, 115 aa, Artificial Sequence; identical to SEQ ID NO: 5
  Synthetic peptide

SEQ ID NO: 35 - DNA, 339 bp, Artificial Sequence
  Synthetic primer
gaaattgtgctgactcagtctccactctccctgcccgtcacccctggagagccggcctccatctcctgcaggtctagtcagagcctcctgtatagtaatggatacaactatttggattggtacctgcagaagccagggaagtctccacaggtcctgatctatttgggttctaatcgggcctccggggtccccgacaggttcagtggcagtggatcaggcacagatttcacactgaaaatcagcagagtggaggctgaggatgttggggtttattactgcatgcaagctctacaaactccgatcaccttcggccaagggacacgactggagattaaacga

SEQ ID NO: 36 - protein, 113 aa, Artificial Sequence
  Synthetic peptide
EIVLTQSPLSLPVTPGEPASISCRSSQSLLYSNGYNYLDWYLQKPGKSPQVLIYLGSNRASGVPDRFSGSGSGTDFTLKISRVEAEDVGVYYCMQALQTPITFGQGTRLEIKR

SEQ ID NO: 37 - DNA, 366 bp, Artificial Sequence
  Synthetic primer
gaggtgcagctggtgcagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccgcttcaccatctccagagacaattccaagaacacgctgtctctgcaaatggacagcctgagacccgaggacacggccgtatattactgtgcgaaaagtcgagatagtgggaactaccttgatgcttttgatttctggggccaagggacaatggtcaccgtctcttca

SEQ ID NO: 38 - protein, 122 aa, Artificial Sequence; identical to SEQ ID NO: 9
  Synthetic peptide

SEQ ID NO: 39 - DNA, 324 bp, Artificial Sequence
  Synthetic primer
gacatccagttgacccagtctccttccaccctgtctgcatctgtaggagacagagtcaccatcacttgccgggccagtcagagtattagtagctggttggcctggtatcagcagaaagcagggaaagctcctaggctcctgatctatgatgcctccactttggaaagtggagtcccatcaaggttcagcggcactggatctgggacatatttcactctcaccatcagcagcctgcagcctgaagattttgcaacttattactgtcaacagtttaatagtttcccgctcactttcggcggagggaccaaggtggagatcaaacga

SEQ ID NO: 40 - protein, 108 aa, Artificial Sequence; identical to SEQ ID NO: 10
  Synthetic peptide

SEQ ID NO: 41 - protein, 5 aa, Artificial Sequence
  Synthetic peptide
GGGGS